(19) United States
(12) Patent Application Publication
(10) Pub. No.: US 2022/0183846 A1
(43) Pub. Date: Jun. 16, 2022

(54) ACETABULAR IMPLANT WITH PREDETERMINED MODULUS AND METHOD OF MANUFACTURING SAME

(71) Applicant: DePuy Ireland Unlimited Company, Ringaskiddy (IE)

(72) Inventors: Fionnan A. McNamara, Carlow (IE); Edward P. Kavanagh, Cork (IE)

(21) Appl. No.: 17/117,166

(22) Filed: Dec. 10, 2020

(51) Int. Cl. A61F 2/34; A61F 2/30

(52) U.S. Cl. CPC A61F 2/34; A61F 2/3094; A61F 2002/3448; A61F 2002/3479; A61F 2310/00017; A61F 2002/30971; A61F 2310/00023; A61F 2310/00029; A61F 2002/30985

(57) ABSTRACT

An acetabular implant can include a predetermined force deflection curve as described herein. The implant can provide individual layers to achieve the predetermined force deflection curve. The acetabular implant can be manufactured using additive manufacturing techniques to achieve the required structures that provide the predetermined force deflection curve.

### BACKGROUND

Examples herein relate generally to the field of Orthopaedics, and, more particularly, to a method of making a 3D printed acetabular implant for a hip prosthesis wherein the implant includes 3D printed structures as part of the body of the implant.

A conventional hip prosthesis can be generally composed of an acetabular implant and a femoral implant. The acetabular portion of the implant typically includes a hemispherical dome-like or cup-like metallic shell secured within the acetabulum and a dome-like or cup-like plastic or ceramic bearing secured within the shell. Various constructions of the shell include an exterior configured to provide a means to anchor the implant into the acetabulum and an interior configured to align and retain the bearing. The bearing typically includes an exterior configured to cooperate with the interior of the shell to align and secure the bearing within the shell and an interior defining an artificial hip socket. In some cases, the exterior of the implant that can be designed to interact with the bone includes a porous structure or coating to promote more rapid bone ingrowth after implantation. The femoral implant typically includes an elongated metallic spike or post at one end and a metallic ball at the other. The post can be generally configured to be anchored into the distal femoral medullary canal and the ball can be generally configured to insert into the artificial socket. The ball of the femoral portion of the implant has freedom to pivotally move within the socket, thereby allowing articulation of the prosthetic joint.

The structure of the acetabular portion of the implant can be designed to provide sufficient rigidity to the implant such that the implant can absorb the normal stresses that the body places on the hip joint. It can be however desirable to provide an implant that also mimics the bone structure and strength of the patient such that the implant forms a more natural part of the patient's anatomy while still maintaining sufficient structural strength.

### SUMMARY

Examples herein provide a 3D printed metal-backed acetabular implant which includes structural components and a method of forming the same.

According to an example, the metal shell includes an inner solid layer and an outer structural layer. The outer structural lay includes a series of structural members such as ribs. The outer surface of the structural members includes a porous layer designed to provide for integration into bone.

According to examples herein, a method can be provided to form a metal-backed acetabular implant according to the configurations indicated above. The method comprises using additive manufacturing techniques to form the metal shell. The additive manufacturing techniques can include, among others, powder bed fusion, direct energy disposition, binder jetting and sheet lamination.

According to examples herein, a method can be provided to form a metal-backed acetabular implant according to the configurations indicated above. The method comprises using a machining formed inner solid layer and forming additional layers onto the machining formed solid core using additive manufacturing techniques. The additive manufacturing techniques can include, among others, powder bed fusion, direct energy disposition, binder jetting and sheet lamination.

Other forms of metal shell can be possible. For instance, the above constructions can also work with, but are not limited to, the shell construction shown in U.S. patent application Ser. No. 11/062,099 entitled INTERNAL ADAPTOR FOR HIP ACETABULAR CAGE, filed on Feb. 18, 2005, herein incorporated by reference in their entirety.

### FIGURES

FIG. 1 shows an example hip prosthesis including an exemplary femoral implant and further including an example acetabular implant.

FIG. 2 shows an isometric view of the example acetabular implant of FIG. 1.

FIG. 3 shows an axial cross-sectional view the shell of the example acetabular implant of FIG. 1.

FIG. 4A shows a sectional view of an example ribbed middle structural layer of the example acetabular implant of FIG. 1.

FIG. 4B shows a sectional view of an example porous middle structural layer of the example acetabular implant of FIG. 1.

FIG. 4C shows a sectional view of an example truss middle structural layer of the example acetabular implant of FIG. 1.

FIG. 5 shows an isometric exploded view of the example implant shell and bearing of FIG. 1.

FIG. 6 shows an isometric view of an example acetabular implant shell with an inner layer formed of interconnected geometric structures.

### DETAILED DESCRIPTION

Like reference numerals refer to like parts throughout the following description and the accompanying drawings. As used herein, the terms “medial,” “medially,” and the like can mean pertaining to the middle, in or toward the middle, and/or nearer to the middle of the body when standing upright. The terms “lateral,” “laterally,” and the like can be used herein as opposed to medial. For example, the medial side of the knee can be the side closest to the other knee and the closest sides of the knees can be medially facing, whereas the lateral side of the knee can be the outside of the knee and can be laterally facing. Further, as used herein the term “superior” can mean closer to the top of the head and/or farther from the bottom of the feet when standing upright. The term “inferior” can be used herein as opposed to superior. For example, the heart can be superior to the stomach and the superior surface of the tongue rests against the palate, whereas the stomach can be inferior to the heart and the palate faces inferiorly toward the tongue. Also, as used herein the terms “anterior,” “anteriorly,” and the like mean nearer the front or facing away from the front of the body when standing upright, as opposed to “posterior,” “posteriorly,” and the like, which mean nearer the back or facing away from the back of the body. Additionally, as used herein, the term “generally hemispherical” can encompass concave and convex geometries suitable for applicable components of prosthetic ball-and-socket type joints such as acetabular and glenoid shells, integuments, bearings, and the like, and, accordingly, includes hemispherical geometries, includes partially spherical geometries that can be more than hemispherical, includes partially spherical geometries that can be less than hemispherical, and includes suitable curved polygonal and geodesic geometries as well.

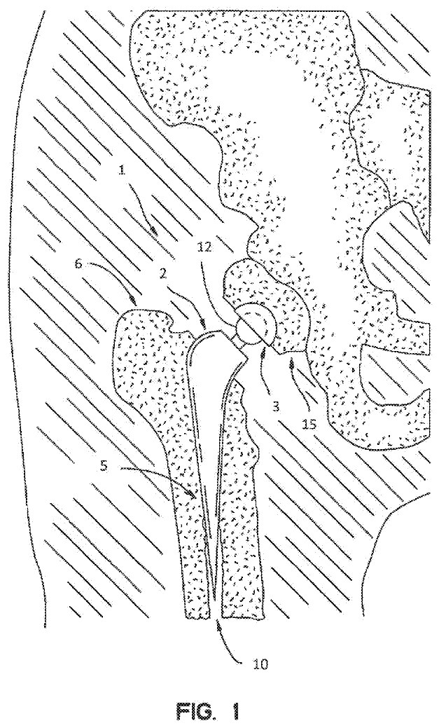

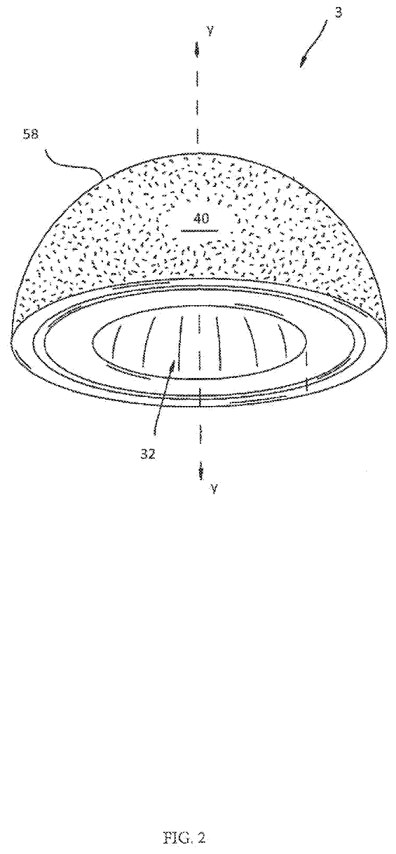

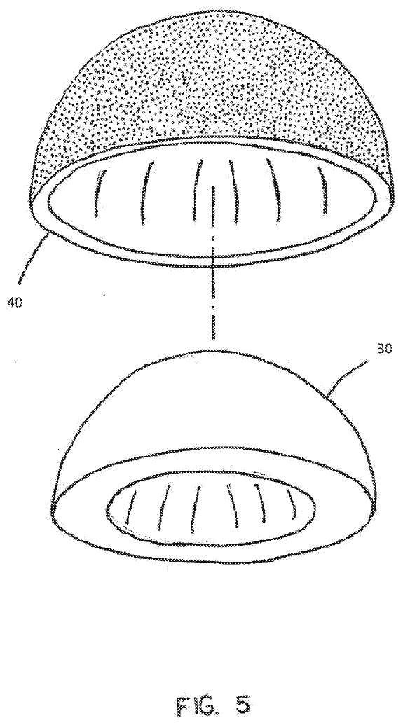

FIG. 1 shows an example hip prosthesis 1 including an example femoral implant 2 and further including an example acetabular implant 3. Among other things, femoral implant 2 can be configured to replace natural hip components (not shown) of a distal femur 6. In the examples herein, femoral implant 2 can be metallic and preferably made from titanium. In alternative embodiments, femoral implant 2 may be made from a cobalt chrome alloy or any other suitable biocompatible material(s). Femoral implant 2 includes a post 5. Among other things, post 5 can be configured as known to anchor into a medullary canal 10 of distal femur 6. Femoral implant 2 also includes a spherical ball 12. Among other things, acetabular implant 3 can be configured to replace natural hip components (not shown) of an acetabulum 15. Accordingly, acetabular implant 3 defines a generally hemispherical artificial hip socket 21 (see FIG. 2 and FIG. 5) that receives ball 12 as known such that ball 12 has suitable pivotal freedom within hip socket 21. Implant 3 can be discussed further below.

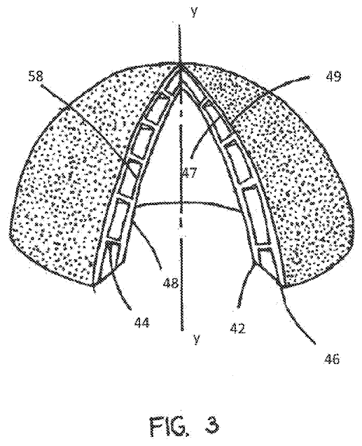

FIG. 2 shows a perspective view of example acetabular implant 3. Acetabular implant 3 includes a dome-like or cup-like acetabular shell 40, and a dome-like or cup-like bearing 30 (see also FIG. 3). Acetabular implant 3 can be configured to be anchored into acetabulum 15 (see FIG. 1) in a known manner. In the examples herein, bearing 30 can be made of a polymeric material such as Ultra High Molecular Weight Polyethylene (UHMWP). In alternative embodiments, bearing 30 may be made from a ceramic material such as alumina or from metallic materials a cobalt chrome alloy or from any other suitable biocompatible material(s). Further the cup-like bearing 30 can be symmetrical about axis y-y and includes a substantially spherical concave inner surface that forms socket 32. Socket 32 can be disposed in a manner that facilitates it in providing a bearing surface for rotational relative motion with the ball 12 of the femoral implant 2 and which provides lateral stability for the ball 12 of femoral implant 2. Bearing 30 further includes a substantially spherical outer surface 34. Acetabular implant 3 further comprises a metallic outer shell 40. Acetabular outer shell 40 can be made of a metallic material such as titanium and can be produced through either an additive manufacturing process, a standard machining process or a hybrid machining and additive manufacturing process. Further, shell 40 can be symmetrical about an axis y-y and includes a generally concave inner surface 48 (see FIG. 3) that can be symmetrical about axis y-y. Acetabular shell 40 can be comprised of an inner solid layer 42, a middle structural layer 44 and an outer layer 46. The inner surface 48 of inner layer 42 forms the inner most surface of the acetabular shell 40. Further, the outer layer 46 of acetabular shell 40 includes a generally hemispherical outer surface 49 facing generally outwardly away from socket 32. In the one or more examples, outer surface 49 can be suitably textured to facilitate fixation in acetabulum 15. Additionally, it can be noted that outer surface 49 may be suitably covered with a porous material (not shown) as known to enhance acetabular fixation of acetabular shell 40 through bone ingrowth.

The inner solid layer 42 of acetabular shell 40 comprises a substantially hemispherical inner surface 48 and an outer surface 58. Inner surface 48 can be disposed such that its radius of curvature can be substantially equal to the radius of curvature of outer surface 34 of bearing 30 such that inner surface 48 forms a support surface for bearing 30 when bearing 30 can be installed in acetabular shell 40. In an example, inner surface 48 and outer surface 58 can be formed such that the thickness T1 of inner layer 42 can be substantially constant. It can be contemplated that in other embodiments, outer surface 58 may not be hemispherical in shape or may be of a hemispherical shape with a different center point than that of inner surface 48 such that the thickness of the inner layer 42 varies. In an example, inner solid layer 42 can be formed such that the thickness T1 of the layer does not impart a significant rigidity to the overall rigidity of the acetabular shell 40.

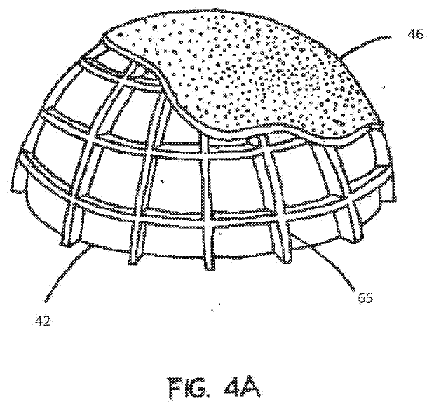

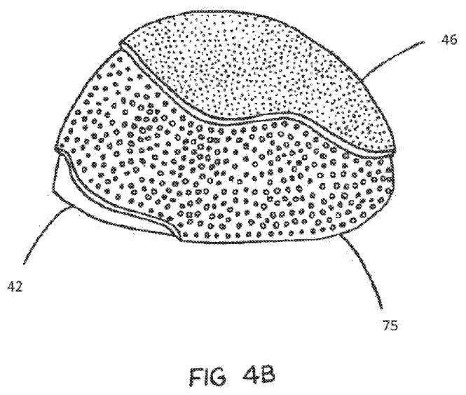

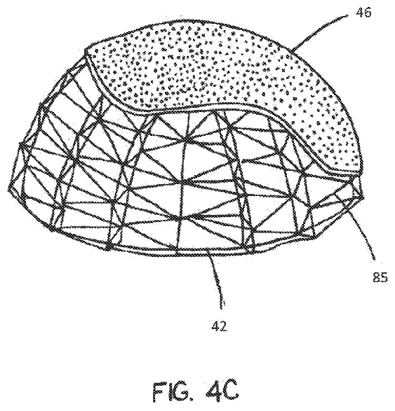

The middle structural layer 44 can be primarily formed from a metallic material such as titanium or other biocompatible metallic materials. Middle structural layer 44 can be integrally attached to outer surface 58 of inner solid layer 42 and can be formed by additive manufacturing techniques as described earlier. Middle structural layer 44 may be formed of a plurality of rib like structures 65 as shown in FIG. 4A, a continuous porous structure 75 as shown in FIG. 4B, truss like structures 85 as shown in FIG. 4C or other structural elements. Middle structural layer 44 can be designed such that the structural layers provide a predetermined force deflection curve for the implant. For instance, the rib like structures 65 may be formed with a width 66 and a height 67 such that the acetabular shell 40 can be significantly more rigid than the bone structures in which the acetabular shell 40 can be placed. Also, rib like structures 65 may be formed discretely from one another or may be integrally attached to one another as shown in FIG. 4A. Alternatively, the height 67 of the rib like structures 65 may be smaller, making the rigidity of the acetabular shell 40 lower. In additional examples, the height 67 of rib like structures 65 may vary over the area of the outer surface 58 of inner solid layer 42. Similarly, the porosity of continuous porous structure 75 and the form of truss like structure 85 may be disposed to create a predetermined rigidity of the acetabular shell 40. Alternatively, various combinations of rib like structure 65, continuous porous structure 75 and truss like structure 85 may be employed. For instance, continuous porous structures 75 may be interspersed between rib like structures 65. The form of middle structural layer 44 can include voids or spaces, such as those that would be created between ribs. Voids such as those described mean that during certain additive manufacturing processes, such as powder bed printing, un-sintered powder will be left in the spaces between the ribs and must be removed prior to encapsulating the middle structural layer 44 in the outer porous layer 46. Process steps for making the acetabular shell 40 will be detailed later in the description.

Outer layer 46 can be formed of a porous or textured metallic material like titanium, titanium alloys, stainless steel, cobalt chrome alloys tantalum or niobium or other biocompatible metals. It can be also contemplated that the outer layer could be formed from a ceramic such as titanium nitride or a carbon material such as silicon carbide Outer layer 46 has an inner surface 47 that can be integrally attached to middle structural layer 44. Outer layer 46 can be formed through additive manufacturing techniques such as those mentioned herein. In an example, outer layer 46 can be formed on acetabular shell 40 and a coating 59 can be subsequently affixed to the outer surface 49. Coating 59 may be comprised of either biocompatible metals or polymers and may be textured, porous or a combination of texture and porosity. Coating 59 and its method of fixation can be accomplished through various techniques including, for example, those described in U.S. Pat. No. 7,368,065 entitled IMPLANTS WITH TEXTURED SURFACE AND METHODS FOR PRODUCING THE SAME, issued on May 6, 2008; U.S. patent application Ser. No. 16/370,599 entitled HYBRID FIXATION FEATURES FOR THREE DIMENSIONAL POROUS STRUCTURES FOR INGROWTH AND METHODS FOR PRODUCING, filed on Mar. 29, 2019; U.S. Pat. No. 10,537,661 entitled ORTHOPEDIC IMPLANT HAVING A CRYSTILINE CALCIUM PHOSPHATE COATING AND METHODS FOR MAKING THE SAME, issued on Jan. 21, 2020; or U.S. Pat. No. 9,308,297 entitled POROUS BIOCOMPATIBLE POLYMER MATERIAL AND METHODS issued on Apr. 12, 2016, each of which is incorporated herein by reference in their entirety. In additional examples, both outer layer 46 and coating 59 can be formed through additive manufacturing techniques as those described herein. In further examples, the outer layer 46 and the coating 59 can be formed through a combination of the prior two embodiments described herein. For instance, some portions of coating 59 may be formed through additive manufacturing techniques while other portions of coating may be applied separately using other manufacturing techniques or coating 59 may be comprised of a first layer applied using additive manufacturing techniques and a second layer applied using other manufacturing techniques. Additionally, outer layer 46 may include openings 52 in its surface to either lighten the overall weight of the acetabular shell 40 or to provide means to remove un-sintered powder used in the additive manufacturing of rib like structures 65 of the middle structural layer 44 described above.

In an example, the entirety of acetabular shell 40 can be formed through additive manufacturing techniques such as powder bed fusion. Powder bed fusion can be an additive manufacturing process and works on the principle that parts can be formed through adding material rather than subtracting it through conventional forming operations such as milling. The powder bed fusion process begins with the creation of a 3D CAD model, which can be numerically ‘sliced’ into discrete layers, each of which can be the thickness of the powder layers applied during manufacturing. For each layer, a scan path can be calculated which defines both the boundary contour and some form of fill sequence, often a raster pattern. An energy source can be then used to melt the powder in prescribed areas, allowing it to bond to adjacent powder particles and form a solid portion of the part. The powders used in this process can be polymeric, metallic or a combination of the two and the methodologies for forming the part include selective laser melting (SLM), selective laser sintering (SLS) direct metal laser sintering (DMLS) and electron beam melting (EBM). Using the energy source, each layer can be sequentially bonded on top of the previous layer. Powder bed fusion processes spread powdered material over the previously joined layer, ready for processing of the next layer such that the manufacturing can be discrete rather than continuous. A hopper supplies the powdered material which can be then spread uniformly over the powder bed build platform area via a roller or blade. The optimal thickness of each layer of spread powder can be dependent on the processing conditions and material used, but values of 25 to 100 μm can be common. Once the part can be fully formed, it can be removed from the powder be fusion system and post processed. Post processing of the part includes removing residual un-melted or un-sintered powder from the part and may further include various heat treatments to improve the structural properties of the part or to stress relieve the part and/or processes to improve the surface finish of the part, including chemical or laser polishing or grit blasting of the part.

Referring to FIG. 5, assembly of acetabular implant 3 can be accomplished by press fitting bearing 30 axially into inner surface 48 to assemble or unite acetabular implant 3. Here, it can be noted that although implant 3 can be fully exploded in FIG. 3 for clarity of exposition, bearing 30 can be preferably pre-operatively press-fitted into inner surface 48. In another example, bearing 30 may be press fitted into an inner metal liner (not shown) by a manufacturer to unite bearing 30 to a metallic support separately from shell 40. Such pre-operative unification of bearing 30 may include temporarily cooling bearing 32 (by immersing it in liquid nitrogen or by any other suitable refrigeration method(s)) immediately prior to press-fitting it into an inner metal liner, followed by allowing bearing 32 to warm or reheat to a normal temperature (and thus un-shrink or re-expand) within the inner metal liner so as to additionally tighten bearing 30 within the inner metal liner.

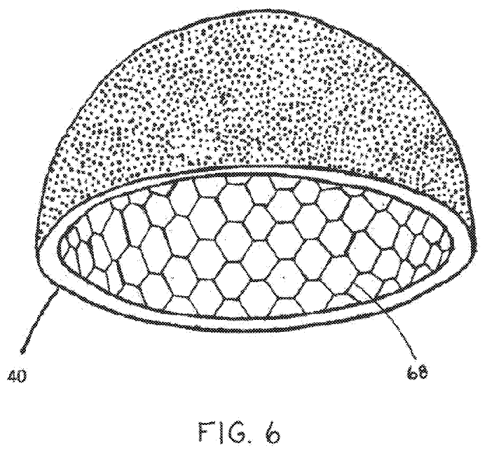

In some alternative embodiments outer layer 46 can be eliminated and middle structural layer 44 can be formed to interact directly with the patient tissue. In another example, outer layer 46 includes a series of large holes evenly or unevenly space along its entire surface. In one or more examples, inner solid layer 42 can be not fully solid and instead can be formed from a series of interconnected hexagonal structures 68 as shown in FIG. 6. These structures could also be formed of other interconnected geometric shapes, such as triangles, squares, octagons or other geometric shapes.

To assemble prosthesis 1, distal femur 6 and acetabulum 15 can be suitably resected, post 5 can be suitably anchored into medullary canal 10, and shell 40 can be suitably anchored into acetabulum 15. Bearing 30 can be rotationally aligned relative to shell 40 and then press-fitted into inner surface 48. Lastly, ball 12 can be inserted into socket 32.

In operation of prosthesis 1, bearing 30 stays coupled to shell 40 within inner surface 48 and pivotal freedom of ball 12 within socket 32 allows articulation of femoral implant 2 relative to acetabular implant 3.

### Example 1

An implant comprising: a femoral implant portion and an acetabular implant portion, said acetabular implant portion comprising a bearing and a shell, comprising a plurality of layers, said plurality of layers including a first solid layer, a second structural layer and a third outer layer wherein said shell further comprises a central axis and wherein said shell is symmetrical about said axis and wherein said second structural layer comprises at least one structural component, said structural component formed by an additive manufacturing process and wherein the dimensions of said structural component are selected to create at least one predetermined force-deflection curve for said shell.

### Example 2

The implant of Example 1, wherein said structural component is selected from a group comprising a rib like structure, a porous solid, a truss like structure or a combination thereof.

### Example 3

The implant of Example 2, wherein said structural component comprises one of a group selected from, a commercially pure titanium, a titanium alloy, a stainless steel alloy, a cobalt chrome alloy, tantalum or niobium.

### Example 4

The implant of example 1, wherein said second structural layer comprises a plurality of structural components and wherein the dimensions of said plurality of structural components are selected to create at least one predetermined force-deflection curve for said shell.

### Example 5

The implant of Example 4, wherein said dimensions of said plurality of structural components create a first predetermined force-deflection curve in a first direction parallel to said axis and wherein said dimensions of said plurality of structural components create a second predetermined force deflection curve in a second direction, wherein said second direction is not parallel to said first direction.

### Example 6

The implant of Example 5, wherein said second direction is perpendicular to said first direction.

### Example 7

The implant of Example 1, wherein each of said at least one predetermined force-deflection curve comprises a slope, said slope being linear over a portion of said predetermined force-deflection curve.

### Example 8

The implant of Example 7, wherein said at least one force deflection curve further comprises a first force-deflection curve comprising a first slope and a second force-deflection curve comprising a second slope, wherein said first slope is different from said second slope.

### Example 9

The implant of Example 8, wherein said linear portion of said slope of said first force-deflection curve is less than said linear portion of said slope of said second force-deflection curve.

### Example 10

The implant of Example 8, wherein said linear portion of said slope of said force-deflection curve is greater than said linear portion of said slope of said second force-defection curve.

### Example 11

The implant of Example 1, wherein said outer layer comprises an inner surface and an outer surface, wherein said inner surface is integrally attached to said second structural layer and wherein said outer surface further comprises a coating.

### Example 12

The implant of Example 11, wherein said coating is selected from a group comprising a coating with a textured outer surface and a porous coating.

### Example 13

An acetabular implant comprising a bearing and a shell, comprising a plurality of layers, said plurality of layers including a first solid layer, a second structural layer and a third outer layer, wherein said shell further comprises a central axis and wherein said shell is symmetrical about said axis and wherein said second structural layer comprises at least one structural component, said structural component formed using an additive manufacturing process and wherein the dimensions of said structural component are selected to create at least one predetermined force-deflection curve for said shell.

### Example 14

The implant of Example 13, wherein said structural component selected from a group comprising, a rib like structure, a porous solid, a truss like structure or a combination thereof.

### Example 15

The implant of Example 14, wherein said structural component comprises one of, a commercially pure titanium, a titanium alloy, a stainless-steel alloy, a cobalt chrome alloy, tantalum or niobium.

### Example 16

The implant of Example 15, wherein said second structural layer comprises a plurality of structural components and wherein the dimensions of said plurality of structural components are selected to create at least one predetermined force-deflection curve for said shell.

### Example 17

The implant of Example 15, wherein said dimensions of said plurality of structural components create a first predetermined force-deflection curve in a first direction parallel to said axis and wherein said dimensions of said plurality of structural components create a second predetermined force deflection curve in a second direction, wherein said second direction is not parallel to said first direction.

### Example 18

The implant of Example 13, wherein each of said at least one predetermined force-deflection curve comprises a slope, said slope being linear over a portion of said predetermined force-deflection curve.

### Example 19

A method for manufacturing an acetabular implant, the method comprising, manufacturing said first solid layer using an additive manufacturing process, manufacturing said second structural layer on said first said solid layer using an additive manufacturing process, and manufacturing said third outer layer on said second structural layer using an additive manufacturing process.

### Example 20

The method of Example 19, wherein said additive manufacturing process is a powder bed fusion process and wherein said additive manufacturing process uses and energy source to selectively melt a powdered metallic material and wherein said further comprises the step of removing all unmelted powdered metallic material from said acetabular implant.

The foregoing description of the invention is illustrative only and is not intended to limit the scope of the invention to the precise terms set forth. Further, although the invention has been described in detail with reference to certain illustrative embodiments, variations and modifications exist within the scope and spirit of the invention as described and defined in the following claims.

We claim:

1. An implant comprising;
a femoral implant portion and;
an acetabular implant portion, said acetabular implant portion comprising;
a bearing and;
a shell, comprising;
a plurality of layers, said plurality of layers including;
a first solid layer;
a second structural layer; and
a third outer layer;
wherein said shell further comprises a central axis and wherein said shell is symmetrical about said axis; and
wherein said second structural layer comprises at least one structural component, said structural component formed by an additive manufacturing process; and
wherein the dimensions of said structural component are selected to create at least one predetermined force-deflection curve for said shell.

2. The implant of claim 1, wherein said structural component is selected from a group comprising; a rib like structure, a porous solid, a truss like structure or a combination thereof.

3. The implant of claim 2, wherein said structural component comprises one of a group selected from, a commercially pure titanium, a titanium alloy, a stainless steel alloy, a cobalt chrome alloy, tantalum or niobium

4. The implant of claim 1, wherein said second structural layer comprises a plurality of structural components and wherein the dimensions of said plurality of structural components are selected to create at least one predetermined force-deflection curve for said shell.

5. The implant of claim 4, wherein said dimensions of said plurality of structural components create a first predetermined force-deflection curve in a first direction parallel to said axis and wherein said dimensions of said plurality of structural components create a second predetermined force deflection curve in a second direction, wherein said second direction is not parallel to said first direction.

6. The implant of claim 5, wherein said second direction is perpendicular to said first direction.

7. The implant of claim 1, wherein each of said at least one predetermined force-deflection curve comprises a slope, said slope being linear over a portion of said predetermined force-deflection curve.

8. The implant of claim 7, wherein said at least one force deflection curve further comprises a first force-deflection curve comprising a first slope and a second force-deflection curve comprising a second slope, wherein said first slope is different from said second slope.

9. The implant of claim 8, wherein said linear portion of said slope of said first force-deflection curve is less than said linear portion of said slope of said second force-deflection curve.

10. The implant of claim 8, wherein said linear portion of said slope of said force-deflection curve is greater than said linear portion of said slope of said second force-defection curve.

11. The implant of claim 1, wherein said outer layer comprises an inner surface and an outer surface, wherein said inner surface is integrally attached to said second structural layer and wherein said outer surface further comprises a coating.

12. The implant of claim 11, wherein said coating is selected from a group comprising a coating with a textured outer surface and a porous coating.

13. An acetabular implant comprising;
a bearing and;
a shell, comprising; a plurality of layers, said plurality of layers including;
a first solid layer;
a second structural layer; and
a third outer layer;
wherein said shell further comprises a central axis and wherein said shell is symmetrical about said axis; and
wherein said second structural layer comprises at least one structural component, said structural component formed using an additive manufacturing process; and
wherein the dimensions of said structural component are selected to create at least one predetermined force-deflection curve for said shell.

14. The implant of claim 13, wherein said structural component selected from a group comprising; a rib like structure, a porous solid, a truss like structure or a combination thereof.

15. The implant of claim 14, wherein said structural component comprises one of, a commercially pure titanium, a titanium alloy, a stainless-steel alloy, a cobalt chrome alloy, tantalum or niobium.

16. The implant of claim 15, wherein said second structural layer comprises a plurality of structural components and wherein the dimensions of said plurality of structural components are selected to create at least one predetermined force-deflection curve for said shell.

17. The implant of claim 15, wherein said dimensions of said plurality of structural components create a first predetermined force-deflection curve in a first direction parallel to said axis and wherein said dimensions of said plurality of structural components create a second predetermined force deflection curve in a second direction, wherein said second direction is not parallel to said first direction.

18. The implant of claim 13, wherein each of said at least one predetermined force-deflection curve comprises a slope, said slope being linear over a portion of said predetermined force-deflection curve.

19. A method for manufacturing an acetabular implant, the method comprising:
manufacturing said first solid layer using an additive manufacturing process;
manufacturing said second structural layer on said first said solid layer using an additive manufacturing process; and
manufacturing said third outer layer on said second structural layer using an additive manufacturing process.

20. The method of claim 19, wherein said additive manufacturing process is a powder bed fusion process and wherein said additive manufacturing process uses and energy source to selectively melt a powdered metallic material and wherein said further comprises the step of removing all unmelted powdered metallic material from said acetabular implant.
